(19) United States
(12) Patent Application Publication
(10) Pub. No.: US 2015/0031542 A1
(43) Pub. Date: Jan. 29, 2015

(54) POLYURETHANE DISPERSIONS FOR SEALING THE TEATS OF THE MAMMARY GLANDS IN MILKING ANIMALS

(71) Applicant: Bayer Intellectual Property GmbH, Monheim (DE)

(72) Inventors: Sebastian Dörr, Dusseldorf (DE); Heike Heckroth, Odenthal (DE); Jan Schoenberger, Haan (DE); Robrecht Froyman, Monheim (DE); Hans-Robert Hehnen, Siegberg (DE); Kristine Fraatz, Burscheid (DE); Hans-Otto Werling, Wuppertal (DE)

(21) Appl. No.: 14/513,306

(22) Filed: Oct. 14, 2014

Related U.S. Application Data
(63) Continuation of application No. 13/515,036, filed on Oct. 23, 2012.

(30) Foreign Application Priority Data
Dec. 12, 2009 (EP) 09015399.0

(51) Int. Cl. A61L 26/00; A61K 9/00

(52) U.S. Cl. CPC A61L 26/0019; A61K 9/0041; A61L 26/0066
USPC 504/360; 424/78.02; 424/500

(57) ABSTRACT

The present invention relates to aqueous polyurethane dispersions for sealing teats of animal mammary glands.

The present invention relates to aqueous polyurethane dispersions for sealing teats of animal mammary glands.

In order to prevent the ingress of microorganisms, especially during the dry phase of cows, the teats of mammary glands are temporarily sealed. This is nowadays done using solutions of polymers in organic solvents. Such systems are for example described in EP 0 973 559 B1 and in WO 02/35931.

Given that contamination of the milk cannot be ruled out, however, the solvents, such as ethyl acetate or tetrahydrofuran, that are contained within the known systems are considered, particularly on contact with livestock whose milk is intended for human consumption, to be objectionable. Moreover, these solvents are classed as “irritant” and their skin compatibility is therefore not good. This sometimes leads to problems in the case of an application period of several weeks. Lastly, the general objections to volatile organic solvents, which consist, for instance, in the possible environmental hazard, are applicable here as well.

An object of the invention, therefore, was to provide a system for sealing teats of animal mammary glands with which there is no risk of milk contamination, which affords good skin compatibility and which is advantageous from the standpoint of environmental hazard.

This object is achieved in accordance with the invention by means of an aqueous polyurethane dispersion for sealing teats of animal mammary glands.

Aqueous polyurethane dispersions as a starting basis for the production of sealing systems have a variety of advantages. In particular, they are safe to use, by virtue of the fact that solvents are very largely absent. Suction withdrawal of solvents is therefore not necessary. Moreover, the use of the aqueous polyurethane dispersions in contact with animals and foodstuffs is unobjectionable.

In principle, all known aqueous polyurethane dispersions can be used. Preference, however, is given to anionically hydrophilized and anionically/nonionically hydrophilized polyurethane dispersions.

Polyurethane dispersions whose use is particularly preferred are obtainable by preparing

A) isocyanate-functional prepolymers from
A1) organic polyisocyanates,
A2) polymeric polyols having number-average molecular weights of 400 to 8000 g/mol, preferably of 400 to 6000 g/mol and more preferably of 600 to 3000 g/mol, and OH functionalities of 1.5 to 6, preferably of 1.8 to 3, more preferably of 1.9 to 2.1, and
A3) optionally hydroxy-functional compounds having molecular weights of 62 to 399 g/mol, and also
A4) optionally isocyanate-reactive, anionic or potentially anionic and/or optionally nonionic hydrophilizing agents,
and

B) then reacting some or all of the free NCO groups of said prepolymers
B1) optionally with amino-functional compounds having molecular weights of 32 to 400 g/mol and
B2) with amino-functional, anionic or potentially anionic hydrophilizing agents
   with chain extension, and dispersing the prepolymers in water before, during or after step B).

Isocyanate-reactive groups are, for example, primary and secondary amino groups, hydroxyl groups or thiol groups.

The aqueous polyurethane dispersions are preferably hydrophilized anionically by means of sulphonate groups and/or carboxylate groups. With particular preference, sulphonate groups exclusively are present for the anionic hydrophilization.

In order to achieve high sedimentation stability, the number-average particle size of the polyurethane dispersions is preferably less than 750 nm, more preferably less than 500 nm, as determined by means of laser correlation spectroscopy.

The polyurethane dispersions preferably possess solids contents of 10% to 70% by weight, more preferably of 30% to 70% by weight, very preferably of 30% to 65% by weight, based on the polyurethane contained therein.

With further preference, these polyurethane dispersions contain less than 5% by weight, more preferably less than 0.2% by weight, based on the total dispersions, of unbound organic amines or of ammonia.

If desired, the prepolymer A) can be converted wholly or partly into the anionic form by admixing of a base before, during or after dispersing.

In order to achieve anionic hydrophilization, it is necessary in A4) and/or B2) to use hydrophilizing agents which have at least one group that is reactive towards NCO groups, such as amino, hydroxyl or thiol groups, and, furthermore, have —COO⁻ or —SO₃⁻ or —PO₃²⁻ as anionic groups and/or their wholly or partly protonated acid forms as potentially anionic groups.

Compounds used for the anionic or potentially anionic hydrophilization in A4) and/or B2) are preferably those which as anionic or potentially anionic functionality have exclusively sulphonic acid and/or sulphonate groups (—SO₃H and/or —SO₃M, with M=alkali metal or alkaline earth metal).

Suitable polyisocyanates of component A1) are the aliphatic, aromatic or cycloaliphatic polyisocyanates with an NCO functionality of greater than or equal to 2 that are known per se to the skilled person.

Examples of such suitable polyisocyanates are 1,4-butylene diisocyanate, 1,6-hexamethylene diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or their mixtures of any desired isomer content, 1,4-cyclohexylene diisocyanate, 4-isocyanatomethyl-1,8-octane diisocyanate (nonane triisocyanate), 1,4-phenylene diisocyanate, 2,4- and/or 2,6-tolylene diisocyanate, 1,5-naphthylene diisocyanate, 2,2′- and/or 2,4′- and/or 4,4′-diphenylmethane diisocyanate, 1,3- and/or 1,4-bis(2-isocyanatoprop-2-yl)benzene (TMXDI), 1,3-bis(isocyanatomethyl)benzene (XDI), and also alkyl 2,6-diisocyanatohexanoates (lysine diisocyanates) having C1-C8-alkyl groups.

Besides the aforementioned polyisocyanates it is also possible to use modified diisocyanates which have a functionality ≧2, having uretdione, isocyanurate, urethane, allophanate, biuret, iminooxadiazinedione or oxadiazinetrione structure, and also mixtures of these, also proportionally.

The compounds in question are preferably polyisocyanates or polyisocyanate mixtures of the aforementioned kind with exclusively aliphatically or cycloaliphatically attached isocyanate groups, or mixtures of these, and with an average NCO functionality of the mixture of 2 to 4, preferably of 2 to 2.6 and more preferably of 2 to 2.4.

Particular preference is given to using, in A1) hexamethylene diisocyanate, isophorone diisocyanate or the isomeric bis(4,4′-isocyanatocyclohexyl)methanes, and also mixtures of the aforementioned isocyanates.

Used in A2) are polymeric polyols having a number-average molecular weight Mₙ of 400 to 8000 g/mol, preferably of 400 to 6000 g/mol and more preferably of 600 to 3000 g/mol. These polyols preferably have an OH functionality of 1.5 to 6, more preferably of 1.8 to 3, very preferably of 1.9 to 2.1.

Polymeric polyols of this kind are the following polyols known per se in polyurethane coatings technology: polyester polyols, polyacrylate polyols, polyurethane polyols, polycarbonate polyols, polyether polyols, polyesterpolyacrylate polyols, polyurethanepolyacrylate polyols, polyurethanepolyester polyols, polyurethanepolyether polyols, polyurethanepolycarbonate polyols and polyesterpolycarbonate polyols. They can be used in A2) individually or in any desired mixtures with one another.

Suitable polyester polyols are also the polycondensates, known per se, of diols and also, optionally, triols and tetraols and of dicarboxylic and also, optionally, tricarboxylic and tetracarboxylic acids or hydroxycarboxylic acids or lactones. In place of the free carboxylic acids it is also possible to use the corresponding polycarboxylic anhydrides or corresponding polycarboxylic esters of lower alcohols to prepare the polyesters.

Examples of suitable diols are ethylene glycol, butylene glycol, diethylene glycol, triethylene glycol, polyalkylene glycols such as polyethylene glycol, and also 1,2-propanediol, 1,3-propanediol, butane-1,3-diol, butane-1,4-diol, hexane-1,6-diol and isomers, neopentyl glycol or neopenyl glycol hydroxypivalate, with preference being given to hexane-1,6-diol and isomers, butane-1,4-diol, neopentyl glycol and neopentyl glycol hydroxypivalate. In addition it is also possible to use polyols such as trimethylolpropane, glycerol, erythritol, pentaerythritol, trimethylolbenzene or trishydroxyethyl isocyanurate.

As dicarboxylic acids it is possible to use phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid itaconic acid, malonic acid, suberic acid, 2-methylsuccinic acid, 3,3-diethylglutaric acid and/or 2,2-dimethylsuccinic acid. As acid source it is also possible to use the corresponding anhydrides.

Where the average functionality of the polyol to be esterified is greater than 2, it is also possible additionally to use monocarboxylic acids such as benzoic acid and hexanecarboxylic acid as well.

Preferred acids are aliphatic or aromatic acids of the aforementioned kind. Particularly preferred are adipic acid, isophthalic acid and phthalic acid.

Hydroxycarboxylic acids which can be used as well as reaction participants in the preparation of a polyester polyol having terminal hydroxyl groups are, for example, hydroxycaproic acid, hydroxybutyric acid, hydroxydecanoic acid, hydroxystearic acid and the like. Suitable lactones are caprolactone, butyrolactone and homologues. Caprolactone is preferred.

Likewise it is possible in A2) to use hydroxyl-group-bearing polycarbonates, preferably polycarbonate diols, having number-average molecular weights Mₙ of 400 to 8000 g/mol, preferably of 600 to 3000 g/mol. They are obtainable by reaction of carbonic acid derivatives, such as diphenyl carbonate, dimethyl carbonate or phosgene, with polyols, preferably diols.

Examples of such diols are ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, dipropylene glycol, polypropylene glycols, dibutylene glycol, polybutylene glycols, bisphenol A and lactone-modified diols of the aforementioned kind.

The diol component preferably contains 40% to 100% by weight of hexanediol, with preference being given more particularly to 1,6-hexanediol and/or hexanediol derivatives. Such hexanediol derivatives are based on hexanediol and as well as terminal OH groups contain ester or ether groups. Derivatives of this kind are obtainable by reacting hexanediol with excess caprolactone or by etherifying hexanediol with itself to give di- or trihexylene glycol.

Instead of or in addition to pure polycarbonate diols it is also possible to use polyetherpolycarbonate diols in A2).

Polycarbonates bearing hydroxyl groups are preferably linear in construction.

In A2) it is likewise possible to use polyether polyols.

Suitability is possessed, for example, by the polytetramethylene glycol polyethers that are known per se in polyurethane chemistry, of the kind obtainable by polymerizing tetrahydrofuran by means of cationic ring opening.

Likewise suitable polyether polyols are the addition reaction products, known per se, of styrene oxide, ethylene oxide, propylene oxide, butylene oxide and/or epichlorohydrin with difunctional or polyfunctional starter molecules. Polyether polyols based on the at least proportional addition of ethylene oxide with difunctional or polyfunctional starter molecules may also be used as component A4) (nonionic hydrophilizing agents).

As suitable starter molecules it is possible to use all of the compounds known from the prior art, such as, for example, water, butyldiglycol, glycerol, diethylene glycol, trimethylolpropane, propylene glycol, sorbitol, ethylenediamine, triethanolamine, 1,4-butanediol.

Preferred components in A2) are polytetramethylene glycol polyethers and polycarbonate polyols and/or mixtures thereof, with polytetramethylene glycol polyethers being particularly preferred.

In A3) it is possible to use polyols of the stated molecular weight range that have up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A (2,2-bis(4-hydroxycyclohexyl)propane), trimethylolpropane, trimethylolethane, glycerol, pentaerythritol, and any desired mixtures thereof with one another.

Also suitable are ester diols of the stated molecular weight range, such as α-hydroxybutyl ε-hydroxycaproic ester, ω-hydroxyhexyl γ-hydroxybutyric ester, adipic acid β-hydroxyethyl ester or terephthalic acid bis(β-hydroxyethyl) ester.

In A3), furthermore, it is also possible to use monofunctional, isocyanate-reactive, hydroxyl-group-containing compounds. Examples of monofunctional compounds of this kind are ethanol, n-butanol, ethylene glycol monobutyl ether, diethylene glycol monomethyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Suitably ionically or potentially ionically hydrophilizing compounds conforming to the definition of component A4) are, for example, monohydroxy- and dihydroxycarboxylic acids, monohydroxy- and dihydroxysulphonic acids, and also monohydroxy- and dihydroxyphosphonic acids and salts thereof, such as dimethylolpropionic acid, dimethylolbutyric acid, hydroxypivalic acid, malic acid, citric acid, glycolic acid, lactic acid, the propoxylated adduct of 2-butenediol and NaHSO₃, described for example in DE-A 2 446 440 (page 5-9, formula I-III).

Suitable nonionically hydrophilizing compounds of component A4) are, for example, polyoxyalkylene ethers which contain at least one hydroxyl, amino or thiol group. Examples are the monohydroxy-functional polyalkylene oxide polyether alcohols that contain on average 5 to 70, preferably 7 to 55, ethylene oxide units per molecule, as are obtainable in conventional manner by alkoxylation of suitable starter molecules (e.g. in Ullmanns Encyclopädie der technischen Chemie, 4th edition, volume 19, Verlag Chemie, Weinheim pp. 31-38). These are either pure polyethylene oxide ethers or mixed polyalkylene oxide ethers, in which case they contain at least 30 mol %, preferably at least 40 mol %, of ethylene oxide units, based on all of the alkylene oxide units present.

Particularly preferred nonionic compounds are monofunctional mixed polyalkylene oxide polyethers which have 40 to 100 mol % of ethylene oxide units and 0 to 60 mol % of propylene oxide units.

Suitable starter molecules for such nonionic hydrophilizing agents are saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethyl-cyclohexane, 3-ethyl-3-hydroxymethyloxetane or tetrahydrofurfuryl alcohol, diethylene glycol monoalkyl ethers, such as, for example, diethylene glycol monobutyl ether, unsaturated alcohols such as allyl alcohol, 1,1-dimethylallyl alcohol or oleyl alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine, and also heterocyclic secondary amines such as morpholine, pyrrolidine, piperidine or 1H-pyrazole. Preferred starter molecules are saturated monoalcohols of the aforementioned kind. Particular preference is given to using diethylene glycol monobutyl ether or n-butanol as starter molecules.

Alkylene oxides that are suitable for the alkoxylation reaction are, in particular, ethylene oxide and propylene oxide, which can be used in any order or else in a mixture in the alkoxylation reaction.

As component B1) it is possible to use organic diamines or polyamines such as, for example, 1,2-ethylenediamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, 4,4-diaminodicyclohexylmethane, hydrazine hydrate, and/or dimethylethylenediamine.

Furthermore it is possible, as component B1), also to use compounds which as well as a primary amino group also contains secondary amino groups or as well as an amino group (primary or secondary) also contain OH groups. Examples of such compounds of primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine.

As component B1) it is also possible, furthermore, to use monofunctional, isocyanate-reactive amine compounds, such as, for example, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, and/or suitable substituted derivatives thereof, amide amines formed from diprimary amines and monocarboxylated acids, monoketimines of diprimary amines, and primary/tertiary amines, such as N,N-dimethylaminopropylamine.

Preference is given to using 1,2-ethylenediamine, bis(4-aminocyclohexyl)methane, 1,4-diaminobutane, isophoronediamine, ethanolamine, diethanolamine and diethylenetriamine.

Suitable anionically hydrophilizing compounds of compound B2) are alkali metal salts of mono-amino- and diaminosulphonic acids. Examples of such anionic hydrophilizing agents are salts of 2-(2-aminoethylamino)ethanesulphonic acid, ethylenediamine-propyl- or -butyl-sulphonic acid, 1,2- or 1,3-propylenediamine-β-ethylsulphonic acid or taurine. Moreover, the salt of cyclohexylaminopropanesulphonic acid (CAPS) from WO-A 01/88006 may be used as an anionic hydrophilizing agent.

Particularly preferred anionic hydrophilizing agents B2) are those which contain sulphonate groups as ionic groups and contain two amino groups, such as the salts of 2-(2-aminoethylamino)ethylsulphonic acid and 1,3-propylenediamine-β-ethylsulphonic acid.

For the hydrophilization it is also possible to use mixtures of anionic and nonionic hydrophilizing agents.

In one preferred embodiment for the preparation of the polyurethane dispersions, components A1) to A4) and B1) to B2) are used in the following amounts, with the individual amounts always adding up to 100% by weight:

5% to 40% by weight of component A1),
55% to 90% by weight of A2),
0.5% to 20% by weight of the sum of components A3) and B1), and
0.1% to 25% by weight of the sum of components A4) and B2), and using, based on the total amounts of components A1) to A4) and B1) to B2), 0.1% to 5% by weight of anionic and/or potentially anionic hydrophilizing agents from A4) and/or B2).

In one particularly preferred embodiment for the preparation of the polyurethane dispersions, components A1) to A4) and B1) to B2) are used in the following amounts, with the individual amounts always adding up to 100% by weight:

5% to 35% by weight of component A1),
60% to 90% by weight of A2),
0.5% to 15% by weight of the sum of components A3) and B1), and
0.1% to 15% by weight of the sum of components A4) and B2), and using, based on the total amounts of components A1) to A4) and B1) to B2), 0.2% to 4% by weight of anionic and/or potentially anionic hydrophilizing agents from A4) and/or B2).

In one especially preferred embodiment for the preparation of the polyurethane dispersions, components A1) to A4) and B1) to B2) are used in the following amounts, with the individual amounts always adding up to 100% by weight:

10% to 30% by weight of component A1),
65% to 85% by weight of A2),
0.5% to 14% by weight of the sum of components A3) and B1), and
0.1% to 13.5% by weight of the sum of components A4) and B2), and using, based on the total amounts of components A1) to A4) and B1) to B2), 0.5% to 3.0% by weight of anionic and/or potentially anionic hydrophilizing agents from A4) and/or B2).

The polyurethane dispersions may be prepared in one or more stages in homogeneous phase or, in the case of multi-stage reaction, partly in disperse phase. Following polyaddition, carried out completely or partially, of A1) to A4), there is a dispersing, emulsifying or dissolving step. This is followed optionally by a further polyaddition or modification in disperse phase.

In this context it is possible to use all of the methods known from the prior art, such as, for example, prepolymer mixing method, acetone method or melt dispersing method. It is preferred to proceed in accordance with the acetone method.

For the preparation by the acetone method, typically, some or all of constituents A2) to A4) and of polyisocyanate component A1), for preparing an isocyanate-functional polyurethane prepolymer, are introduced as an initial charge, and are optionally diluted with a solvent which is miscible with water but is inert towards isocyanate groups, and heated to temperatures in the range from 50 to 120° C. In order to accerate the isocyanate addition reaction it is possible to use the catalysts known in polyurethane chemistry.

Suitable solvents are the typical aliphatic, keto-functional solvents such as acetone, 2-butanone, which can be added not only at the beginning of the preparation but also, if desired, in portions later on as well. Preferred are acetone and 2-butanone; particularly preferred is acetone. The addition of other solvents without isocyanate-reactive groups is also possible, but not preferred.

Subsequently, any constituents from A1) to A4) not added at the beginning of the reaction are metered in.

In the preparation of the polyurethane prepolymer from A1) to A4), the amount-of-substance ratio of isocyanate groups to isocyanate-reactive groups is generally 1.05 to 3.5, preferably 1.1 to 3.0, more preferably 1.1 to 2.5.

The reaction of components A1) to A4) to give the prepolymer takes place partially or completely, but preferably completely. In this way, polyurethane prepolymers containing free isocyanate groups are obtained, in bulk or in solution.

Thereafter, in a further process step, if it has not yet taken place or has taken place only partially, the prepolymer obtained is dissolved using aliphatic ketones such as acetone or 2-butanone.

In the neutralizing step for the partial or complete conversion of potentially anionic groups into anionic groups, bases are used such as tertiary amines, examples being trialkylamines having 1 to 12, preferably 1 to 6, C atoms in each alkyl radical, or alkali metal bases such as the corresponding hydroxides.

Examples thereof are trimethylamine, triethylamine, methyldiethylamine, tripropylamine, N-methylmorpholine, methyldiisopropylamine, ethyldiisopropylamine and diisopropylethylamine. The alkyl radicals may, for example, also carry hydroxyl groups, as in the case of the dialkyl-monoalkanolamines, alkyldialkanolamines and trialkanolamines. As neutralizing agents it is also possible to use inorganic bases, such as aqueous sodium hydroxide, lithium hydroxide and potassium hydroxide.

Preference is given to sodium hydroxide, lithium hydroxide or potassium hydroxide; particularly preferred are sodium hydroxide, lithium hydroxide or potassium hydroxide. With very particular preference, the sodium, lithium or potassium ions are already attached as cation to anionically functionalized building blocks.

The amount of substance of the bases is generally between 50 and 125 mol %, preferably between 70 and 100 mol %, of the amount of substance of the acid groups to be neutralized. The neutralization may also take place simultaneously with the dispersing, with the dispersing water already containing the neutralizing agent.

In the chain extension in stage B), NH₂— and/or NH-functional components are reacted with the remaining isocyanate groups of the prepolymer. The chain extension/termination is carried out preferably prior to the dispersing in water.

Suitable components for the chain extension are organic diamines or polyamines B1) such as, for example, ethylenediamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, diaminodicyclohexylmethane and/or dimethylethylenediamine.

It is also possible, furthermore, to use compounds B1) which as well as a primary amino group also have secondary amino groups or as well as an amino group (primary or secondary) also have OH groups. Examples thereof are primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine, used for the chain extension or termination.

For the chain termination it is usual to use amines B1) having an isocyanate-reactive group, such as methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, and/or suitable substituted derivatives thereof, amide amines formed from diprimary amines and monocarboxylic acids, monoketimines of diprimary amines, and primary/tertiary amines, such as N,N-dimethyl-aminopropylamine.

Where chain extension is carried out using anionic hydrophilizing agents meeting the definition B2) having NH₂— or NH-groups, the chain extension of the prepolymers takes place preferably before the dispersing.

The degree of chain extension, in other words the equivalents ratio of NCO-reactive groups of the compounds used for chain extension and chain termination to free NCO groups of the prepolymer, is situated in general between 40 and 150%, preferably between 50 and 120%, more preferably between 60 and 120%.

The aminic components B1) and B2) can be used optionally in diluted form in the process of the invention, individually or in mixtures, with any sequence of the addition being possible in principle.

If water is among the diluents used, the diluent content of the component used in B) for chain extension is preferably 40% to 95% by weight.

The dispersing takes place preferably following the chain extension. For this purpose, either the dissolved and chain-extended polyurethane polymer is introduced into the dispersing water, optionally with strong shearing, such as vigorous stirring, for example, or, conversely, the dispersing water is stirred into the chain-extended polyurethane polymer solutions. It is preferred to add the water to the dissolved, chain-extended polyurethane polymer.

The solvent still present in the dispersion after the dispersing step is typically removed subsequently by distillation. Removal actually during the dispersing is likewise possible.

The residual organic solvents content of the polyurethane dispersions is typically less than 2% by weight, preferably less than 1% by weight, based on the total dispersion.

The pH of the polyurethane dispersions is typically less than 8.0, preferably less than 7.5 and more preferably between 5.5 and 7.5.

The polyurethane dispersions typically contain at least 10% by weight of polyurethane, based on the solids fraction of all of the film-forming polymers present in the dispersion. Preferably, however, at least 50% by weight, more preferably at least 90% by weight, very preferably at least 95% by weight, and more particularly preferably 100% by weight of polyurethane is present as film-forming polymer. In this context it has been found that the water resistance of the seals obtained increases with the polyurethane content, thus providing improved durability.

If polyurethane is not used exclusively as film-forming polymer, then other polymer dispersions, furthermore, may be used as well, being based, for example, on polyesters, poly(meth)acrylates, polyepoxides, polyvinyl acetates, polyethylene, polystyrene, polybutadienes, polyvinyl chloride and/or corresponding copolymers.

Besides the polymer dispersions, the polyurethane dispersions may in addition also comprise auxiliaries and adjuvants. Examples of such auxiliaries and adjuvants are crosslinkers, thickeners, thixotropic agents, stabilizers, antioxidants, light stabilizers, emulsifiers, surfactants, plasticizers, pigments, fillers and flow control agents.

The application of the polyurethane dispersions of the invention may take place by any forms of application that are known per se; examples include dipping, brushing, pouring or spraying. Particularly preferred are spraying and dipping; dipping is especially preferred.

The polyurethane dispersions are typically dried in the air.

A multi-coat application with drying steps in between if desired is also possible in principle.

The films obtained after drying typically have a thickness of 0.1 to 1500 μm, preferably 1 to 500 μm more preferably 5 to 200 μm, very preferably 50 to 150 μm.

Furthermore, the polyurethane dispersions may be admixed with active ingredients or applied in combination with active ingredients. In the text below, active ingredients are defined as elements or chemical compounds which have an action on living systems, more particularly prions, viruses, bacteria, cells, fungi and organisms.

Examples are active biocidal ingredients which act, for example, pesticidally, fungicidally, algicidally, insecticidally, herbicidally, spermicidally, parasiticidally, antibacterially (destroying bacteria), bacteriostatically, antibiotically, antimycotically (destroying fungi), antivirally (destroying viruses), virostatically and/or antimicrobially (destroying microbes). Active ingredient combinations as well, and combination with, for example, auxiliaries, binders, neutralizing agents or additives, are possible. Other active ingredients and combinations as well can be employed, examples being active ingredients from the area of human medicine or veterinary medicine. It is preferred to add at least one additive having an antibacterial, bacteriostatic or antibiotic action.

### EXAMPLES

Unless indicated otherwise, all percentages are by weight.

Unless noted otherwise, all analytical measurements are based on temperatures of 23° C.

The solids contents were determined in accordance with DIN-EN ISO 3251.

NCO contents, unless expressly stated otherwise, were determined volumetrically in accordance with DIN-EN ISO 11909.

Monitoring for free NCO groups was carried out by means of IR spectroscopy (band at 2260 cm⁻¹).

The reported viscosities were determined by means of rotational viscometry in accordance with DIN 53019 at 23° C. using a rotational viscometer from Anton Paar Germany GmbH, Ostfildern, DE.

The average particle sizes (the parameter stated is the numerical average) of the polyurethane dispersions were determined by means of laser correlation spectroscopy (instrument: Malvern Zetasizer 1000, Malvern Instr. Limited).

### Substances Used and Abbreviations:

Diaminosulphonate: NH₂—CH₂CH₂—NH—CH₂CH₂—SO₃Na (45% strength in water)
Desmophen® 2020/C2200: Polycarbonate polyol, OH number 56 mg KOH/g, number-average molecular weight 2000 g/mol (Bayer MaterialScience AG, Leverkusen, DE)
PolyTHF® 2000: Polytetramethylene glycol polyol, OH number 56 mg KOH/g, number-average molecular weight 2000 g/mol (BASF AG, Ludwigshafen, DE)
PolyTHF® 1000: Polytetramethylene glycol polyol, OH number 112 mg KOH/g, number-average molecular weight 1000 g/mol (BASF AG, Ludwigshafen, DE)

Polyether LB 25: Monofunctional polyether based on ethylene oxide/propylene oxide, number-average molecular weight 2250 g/mol, OH number 25 mg KOH/g (Bayer MaterialScience AG, Leverkusen, DE)

### Example 1

### Polyurethane Dispersion 1

Amounts of 987.0 g of PolyTHF® 2000, 375.4 g of PolyTHF® 1000, 761.3 g of Desmophen® C2200 and 44.3 g of Polyether LB 25 were heated to 70° C. in a standard stirring apparatus. Subsequently at 70° C. over the course of 5 minutes a mixture of 237.0 g of hexamethylene diisocyanate and 313.2 g of isophorone diisocyanate was added and the mixture was stirred at 120° C. until the theoretical NCO value was reached. The finished prepolymer was dissolved with 4830 g of acetone, in the course of which it was cooled to 50° C., and then a solution of 25.1 g of ethylenediamine, 116.5 g of isophoronediamine, 61.7 g of diaminosulphonate and 1030 g of water was metered in over the course of 10 minutes. The subsequent stirring time was 10 minutes. Dispersion was then carried out by addition of 1250 g of water. This was followed by the removal of the solvent by vacuum distillation. The residual acetone content was below 1% by weight, based on the finished dispersion.

The white dispersion obtained had the following properties:

Solids content: 61%
Particle size (LCS): 312 nm
Viscosity (viscometer, 23° C.): 241 mPas

### pH (23° C.): 6.02

### Example 2

### Polyurethane Dispersion 2

Amounts of 450 g of PolyTHF® 1000 and 2100 g of PolyTHF® 2000 were heated to 70° C. Subsequently at 70° C. over the course of 5 minutes a mixture of 225.8 g of hexamethylene diisocyanate and 298.4 g of isophorone diisocyanate was added and the mixture was stirred at 100-115° C. until the NCO value was below the theoretical NCO value. The finished prepolymer was dissolved with 5460 g of acetone at 50° C., and then a solution of 29.5 g of ethylenediamine, 143.2 g of diaminosulphonate and 610 g of water was metered in over the course of 10 minutes. The subsequent stirring time was 15 minutes. Dispersion was then carried out by addition of 1880 g of water over the course of 10 minutes. This was followed by the removal of the solvent by vacuum distillation, to give a storage-stable dispersion. The residual acetone content was below 1% by weight, based on the finished dispersion.

Solids content: 56%
Particle size (LCS): 276 nm
Viscosity: 1000 mPas

### pH (23° C.): 7.15

### Example 3

### Polyurethane Dispersion 3

An amount of 1649.0 g of a polyester formed from adipic acid, hexanediol and neopenyl glycol, having an average molecular weight of 1700 g/mol, was heated to 65° C. Subsequently at 70° C. over the course of 5 minutes 291.7 g of hexamethylene diisocyanate was added and the mixture was stirred at 100-115° C. until the NCO value was below the theoretical NCO value. The finished prepolymer was dissolved with 3450 g of acetone at 50° C., and then a solution of 16.8 g of ethylenediamine, 109.7 g of diaminosulphonate and 425 g of water was metered in over the course of 15 minutes. The subsequent stirring time was 15 minutes. Dispersion was then carried out by addition of 1880 g of water over the course of 10 minutes. This was followed by the removal of the solvent by vacuum distillation, to give a storage-stable dispersion.

Solids content: 42%
Particle size (LCS): 168 nm
Viscosity: 425 mPas
pH: 7.07

### Example 4

### PU Dispersion 4

Amounts of 82.5 g of PolyTHF® 1000, 308 g of PolyTHF® 2000 and 10.0 g of 2-ethylhexanol were heated to 70° C. Subsequently at 70° C. over the course of 5 minutes a mixture of 41.4 g of hexamethylene diisocyanate and 54.7 g of isophorone diisocyanate was added and the mixture was stirred at 100-125° C. until the NCO value was below the theoretical NCO value. The finished prepolymer was dissolved with 880 g of acetone at 50° C., and then a solution of 3.8 g of ethylenediamine, 4.6 g of isophoronediamine, 26.3 g of diaminosulphonate and 138 g of water was metered in over the course of 10 minutes. The subsequent stirring time was 15 minutes. Dispersion was then carried out by addition of 364 g of water over the course of 10 minutes. This was followed by the removal of the solvent by vacuum distillation, to give a storage-stable dispersion.

Solids content: 49%
Particle size (LCS): 181 nm
Viscosity: 1300 mPas
pH: 7.22

### Example 5

### Preparation of the Coating Solutions

The PU dispersions prepared in Examples 1 to 4 were each adjusted with water and/or thickener (Borchigel ALA) to a viscosity of 300 to 500 mPas (23° C.) and coloured with blue food dye (Dualcert blue No. 1, SENSIENT COLORS, UK).

### Example 6

### Sealing of Teats

The teat sealing experiments were carried out on dairy cows during the hormonally induced drying-off phase. For these experiments, one at a time of the coating solutions prepared in Example 5 was applied as a film, by dipping, to the teats of an udder of a cow. Drying took place in the air. The coatings obtained sealed the teats. Surprisingly, moreover, they exhibited a durability of up to 3 days, even under the damp conditions in a cow shed—in other words, within this time, the seal remained intact. The systems of the invention were therefore equal to standard commercial solvent-borne systems such as DryFlex from DeLaval or calgodip T-Hexx Dry from Kleancare Hygiene GmbH, for example, while contrasting with the standard commercial systems in not employing (volatile) organic solvents.

1-15. (canceled)

16. A method for sealing a teat of an animal mammary gland comprising applying a coating of a polyurethane dispersion to the teat of the animal mammary gland, wherein the polyurethane dispersion is obtained by preparing
A) isocyanate-functional prepolymers from
A1) organic polyisocyanates,
A2) polymeric polyols having number-average molecular weights of 400 to 8000 g/mol, and OH functionalities of 1.5 to 6, and
A3) optionally hydroxy-functional compounds having molecular weights of 62 to 399 g/mol, and also
A4) optionally isocyanate-reactive, anionic or potentially anionic and/or optionally nonionic hydrophilizing agents,
and
B) then reacting some or all of the free NCO groups of said prepolymers
B1) optionally with amino-functional compounds having molecular weights of 32 to 400 g/mol and
B2) with amino-functional, anionic or potentially anionic hydrophilizing agents
with chain extension, and dispersing the prepolymers in water before, during or after step B).

17. The method of claim 16, wherein components A1) to A4) and B1) to B2) are used in the following amounts, wherein the sum of the individual amounts total 100% by weight:
from 5% to 40% by weight of component A1),
from 55% to 90% by weight of A2),
from 0.5% to 20% by weight of the sum of components A3) and B1), and
from 0.1% to 25% by weight of the sum of components A4) and B2),
and using, based on the total amounts of components A1) to A4) and B1) to B2), 0.1% to 5% by weight of anionic and/or potentionally anionic hydrophilizing agents from A4) and/or B2).

18. The method of claim 16, wherein the number-average particle size of the particles in the polyurethane dispersions, as determined by means of laser correlation spectroscopy, is less than 750 nm.

19. The aqueous polyurethane dispersion of claim 16, wherein the polyurethane dispersion has solids contents of 10% to 70% by weight, based on the polyurethane contained therein.

20. The aqueous polyurethane dispersion of claim 16, wherein the polyurethane dispersion contains less than 5% by weight, based on the total dispersion, of unbound organic amines.

21. The aqueous polyurethane dispersion of claim 16, wherein there are no volatile amines and no ammonia in the polyurethane dispersion.

22. The method of claim 16, wherein in the polyurethane dispersion there is less than 2% by weight of organic solvents.

23. The method of claim 16, wherein there is at least one active biocidal ingredient in the polyurethane dispersion.

24. The method of claim 16, wherein the active biocidal ingredient is pesticidal, fungicidal, algicidal, insecticidal, herbicidal, spermicidal, parasiticidal, antibacterial, bacteriostatic, antibiotic, antimycotic, antiviral, virostatic and/or antimicrobial.

25. The method of claim 16, wherein the coating is applied to teats of the udder of dairy cows.

26. The method of claim 26, wherein the polyurethane dispersion is applied to the teats during the dry phase of the dairy cows.

27. The method of claim 18, wherein A2) are polymeric polyols having number-average molecular weights of 400 to 6000 g/mol, and OH functionalities of 1.8 to 3.

28. The aqueous polyurethane dispersion of claim 18, wherein A2) are polymeric polyols having number-average molecular weights of 600 to 3000 g/mol, and OH functionalities of 1.9 to 2.1.

29. The method of claim 20, wherein the number-average particle size of the particles in the polyurethane dispersions, as determined by means of laser correlation spectroscopy, is less than 500 nm.

30. The method of claim 19, wherein the polyurethane dispersion has solids contents of 30% to 70% by weight, based on the polyurethane contained therein.

31. The method of claim 19, wherein the polyurethane dispersion has solids contents of 30% to 65% by weight, based on the polyurethane contained therein.

32. The method of claim 20, wherein the polyurethane dispersion contains less than 0.2% by weight, based on the total dispersion, of unbound organic amines.

33. The method of claim 22, wherein in the polyurethane dispersion there is less than 1.5% by weight of organic solvents.

34. The method of claim 16, wherein the polyurethane dispersion is anionically and/or nonionically hydrophilized.

35. The method of claim 16, wherein the teat of the animal mammary gland is sealed for up to 3 days.
